(19) United States
(12) Patent Application Publication
(10) Pub. No.: US 2008/0082074 A1
(43) Pub. Date: Apr. 3, 2008

(54) Absorbent article with refastenable and adjustable side panels

(76) Inventors: Arginnys Soto, Yardley, PA (US); Lanying Wu, Paoli, PA (US); Stacy Driskell, Royersford, PA (US); Nadine Clark, Quakertown, PA (US)

Correspondence Address: GOSZ AND PARTNERS LLP, ONE STATE STREET, BOSTON, MA, 02109, US

(21) Appl. No.: 11/540,100

(22) Filed: Sep. 29, 2006

(51) Int. Cl. A61F 13/15

(52) U.S. Cl. 604/385.26; 604/389

(57) ABSTRACT

An absorbent article comprising a single side panel permanently attached to one of the front waist region and the back waist region is provided. The side panel is non-contoured and elastic, and can be refastened or adjusted to the body to maintain the article in a pant configuration.

### BACKGROUND OF THE INVENTION

The present invention relates to disposable absorbent articles. More particularly, the present invention relates to a pant-like absorbent article with refastenable, non contoured side panels and improved body fit.

Disposable absorbent articles, such as disposable diapers, training pants, sanitary napkins, protective underwear, and the like are well known. Such articles are provided in various sizes, shapes and conformations to meet the individual requirements of consumers. Such articles are made of an absorbent chassis that includes a body facing top sheet, a non-body facing back sheet and an absorbent core. The top sheet is made from a fluid permeable material that is soft to feel and non-irritable to the skin so as not to cause discomfort to the wearer. The back sheet is made from a fluid impermeable material so as to prevent leakage of body exudates that passes through the absorbent core. The absorbent core is typically made of an absorbent material such as a super absorbent polymer (SAP), in combination with paper fluff. The absorbent articles also include some form of attachment system to fit the article about the wearer.

It is desired that absorbent articles provide a close, comfortable fit about the wearer and contain body exudates. Moreover, it is desirable that such absorbent articles, after being soiled, can be removed from the wearer by a care giver in a convenient and clean manner. In addition, it is desirable that the article can be adjustable and refastenable to provide for a better fit for the wearer.

An absorbent article is therefore required that is easy to manufacture and has a refastenable mechanism that improves body fit and reduces the likelihood of leakage.

Accordingly, it is an objective of the present invention to provide an absorbent article having a comfortable fit about the wearer that can be removed from the wearer in a convenient and clean manner.

It is another objective of the present invention to provide an absorbent article with a refastenable mechanism that allows the absorbent article to be easily adjusted during use.

### SUMMARY OF THE INVENTION

In order to achieve the abovementioned objectives, the invention provides an absorbent article having an absorbent chassis comprising a top sheet, a back sheet and an absorbent core disposed between the top sheet and the back sheet. The absorbent chassis defines a front waist region, a back waist region and a crotch region that extends between and interconnects the front and back waist regions.

A non contoured side panel is permanently attached to and extends away from the lateral edges of the back waist region of the absorbent chassis. The absorbent article has at least one fastening component attached to the front waist region of the absorbent chassis. A side panel extending from the back waist region of the absorbent chassis is engaged to the fastening component on the front waist region to secure the absorbent article on a wearer.

### BRIEF DESCRIPTION OF DRAWINGS

These and other features and advantages of the present invention will be appreciated, as they become better understood by reference to the following Detailed Description when considered in connection with the accompanying drawings:

FIG. 1 is a top view of a pant-like, disposable diaper according to an exemplary embodiment of the present invention;

FIG. 2 shows the repositioning of the diaper along a line that divides crotch region of the diaper into two halves;

FIG. 3 shows a front view of the diaper upon folding along the line dividing the crotch region of the diaper in two halves;

FIG. 4 shows a side view of the diaper in accordance with an exemplary embodiment of the present invention;

FIG. 5 is an alternate embodiment of the present invention with side panel attached to the front waist region; and

FIGS. 6A, 6B and 6C are detailed cross-sectional views of preferred embodiments of the diaper that show the spatial relationship between the various parts of the diaper.

### DETAILED DESCRIPTION OF THE INVENTION

As used herein, the terms “absorbent article”, or simply “article”, refer to garments that absorb and contain body fluids and other body exudates. More specifically, these terms refer to garments that are placed against or in proximity to the body of a wearer to absorb and contain the various exudates discharged from the body. A non-exhaustive list of examples of absorbent garments includes pantiliners, sanitary napkins, feminine hygiene products, diapers, diaper covers, disposable diapers, training pants, and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent articles, without limitation, whether disposable or otherwise. The absorbent article of the present invention is optionally a diaper, pantiliner, sanitary napkin, feminine hygienic garment, incontinent brief, training pant, diaper holder, or a combination thereof.

The embodiments described herein provide an exemplary structure of a disposable, pant-like diaper article which is adapted to be worn by infants about the lower torso. However this is not intended to limit the scope of the claimed invention. The invention will be understood to encompass, without limitation, all classes, and types of absorbent articles, including those described herein. In the foregoing specification, specific embodiments of the present invention have been described. However, one of ordinary skill in the art will appreciate that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

The present invention relates to the configuration of an absorbent article such as a diaper, training pant, sanitary pad, and pantiliner having a single side panel for fastening around the waist region of a wearer. More particularly, the present invention relates to a pant-like disposable diaper that has a front waist region, a back waist region, and a crotch region positioned between the front waist and rear waist region. Further, the diaper is provided with a single side panel which is permanently attached to one of the front waist region and the back waist region.

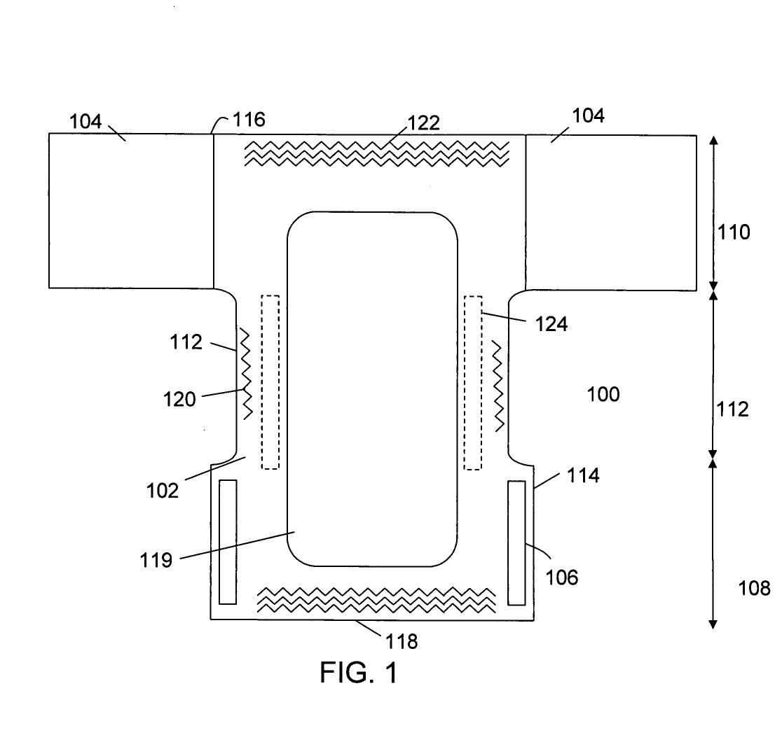

With reference to FIG. 1, a top view of a pant-like disposable diaper 100 according to an exemplary embodiment of the present invention is provided. Diaper 100 comprises an absorbent chassis 102, a single side panel 104 and a fastening component 106 attached to absorbent chassis 102. Side panel 104 is non-contoured and stretchable, and can be refastened or adjusted to the body of the wearer to maintain the product in a pant configuration. Absorbent chassis 102 defines a front waist region 108, a back waist region 110, a crotch region 112 which extends between and connects the front and back waist regions 108 and 110.

In the embodiment shown in FIG. 1, side panel 104 is attached to back waist region 110 and fastening component 106 is attached to front waist region 108 of absorbent chassis 102. Diaper 100 includes a pair of laterally opposed side edges, 112 and 114 and a pair of longitudinally opposed waist edges 116 and 118.

Front waist region 108 comprises the portion of diaper 100 which, when worn, is positioned on the front of the wearer while back waist region 110 comprises the portion of diaper 100 which, when worn, is positioned on the back of the wearer. Crotch region 112 comprises the portion of diaper 100 which includes an absorbent core 119. When diaper 100 is worn, crotch region 112 is positioned between the legs of the wearer. Side panel 104 comprise the portions of the diaper which, when worn, are positioned on the side hip regions of the wearer. The laterally opposed side edges, 112 and 114 and side panel 104 of diaper 100 generally define leg openings which may be curvilinear. The waist edges 116 and 118 of diaper 100 and side panel 104 are configured to encircle the waist of the wearer when worn and provide a waist opening when fastened. Diaper 100 may further include a pair of leg elastics 120, a pair of waist elastics 122 and containment flaps 124, as are known to those skilled in the art. Leg elastics 120 and waist elastics 122 are generally adapted to fit about the legs and waist of a wearer in use, to maintain a contacting relationship with the wearer. Containment flaps 124 help reduce the leakage of body exudates from diaper 100. It should be recognized that individual components of diaper 100 may be optional depending upon the intended use of diaper 100.

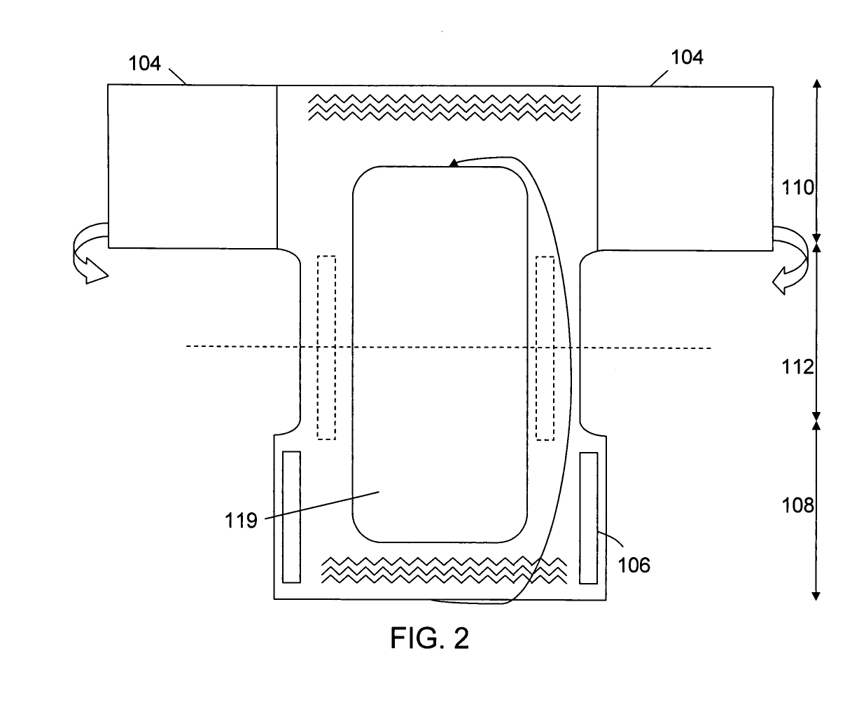

FIG. 2 shows the repositioning of diaper 100 along a line that divides crotch region 112 in two halves. Upon repositioning, front waist region 108 gets positioned on front waist of a wearer, when worn and crotch region 112 covers the lower torso of the wearer. Side panel 104 is folded inwardly so as to encircle the side hips of the wearer.

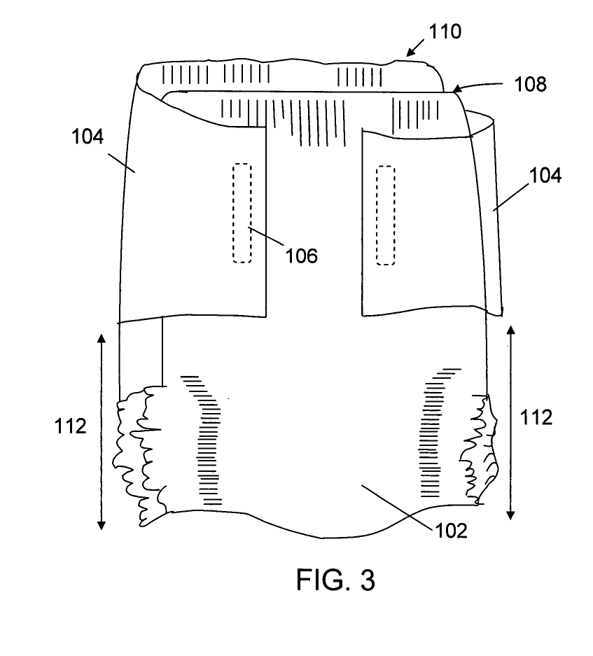

FIG. 3 shows a front view of diaper 100 upon folding along the line dividing crotch region 112 in two halves. Side panel 104 is non-contoured and elastic, and can be refastened or adjusted to the body to maintain the article in a pant configuration. Side panel 104 may be permanently attached to back waist region 110 of absorbent chassis 102 using attachment means such as adhesive, thermal, or ultrasonic bonding. In a preferred embodiment, side panel 104 is permanently attached to absorbent chassis 102 using ultrasonic bonding without creating any vent holes for improved comfort for the wearer. Fastening component 106 attached to front waist region 108 of absorbent chassis 102 is used for releasably fastening side panel 104 to front waist region 108 of absorbent chassis 102.

In one embodiment, fastening component 106 is attached to side panel 104 through a fastener grip provided on side panel 104. Fastening component 106 may be provided by using conventional means, well known to those skilled in the art, using any type of bonding such as adhesive, cohesive, or mechanical systems. Absorbent articles including such releasable systems and methods of making such are further described in a U.S. patent application of Elsberg and assigned U.S. Ser. No. 09/100,574, and U.S. Pat. No. 6,036,805 issued to McNichols, the respective disclosures of which are hereby incorporated by reference in their entireties.

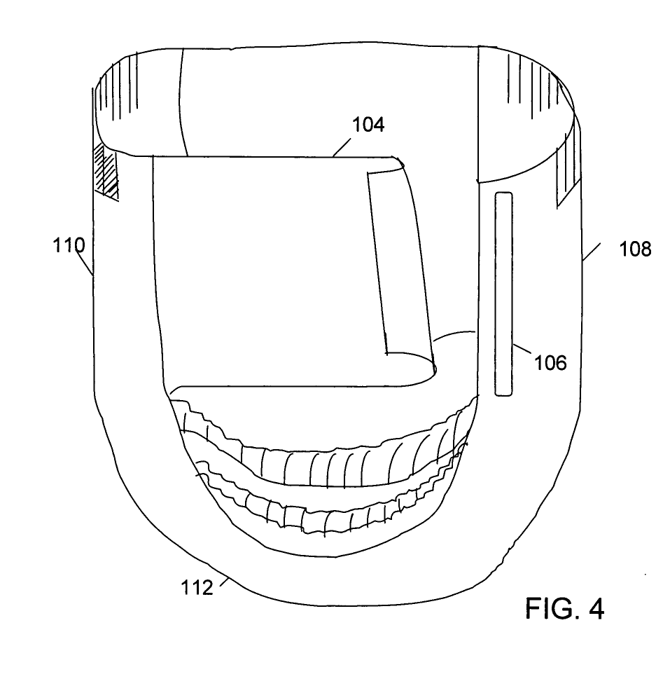

FIG. 4 shows a side view of diaper 100. As shown, side panel 104 attached to back waist region 110 of diaper 100 may be releasably fastened to fastening component 106 provided on front waist region 108 to maintain the article in a pant configuration.

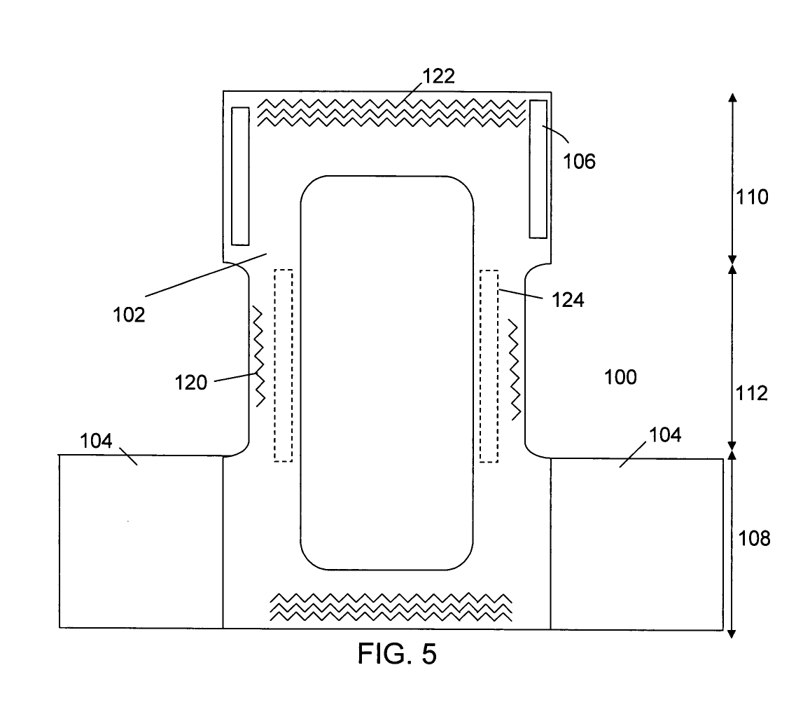

FIG. 5 is an alternate embodiment of the present invention with side panel 104 attached to front waist region 108 and fastening component 106 provided on back waist region 110.

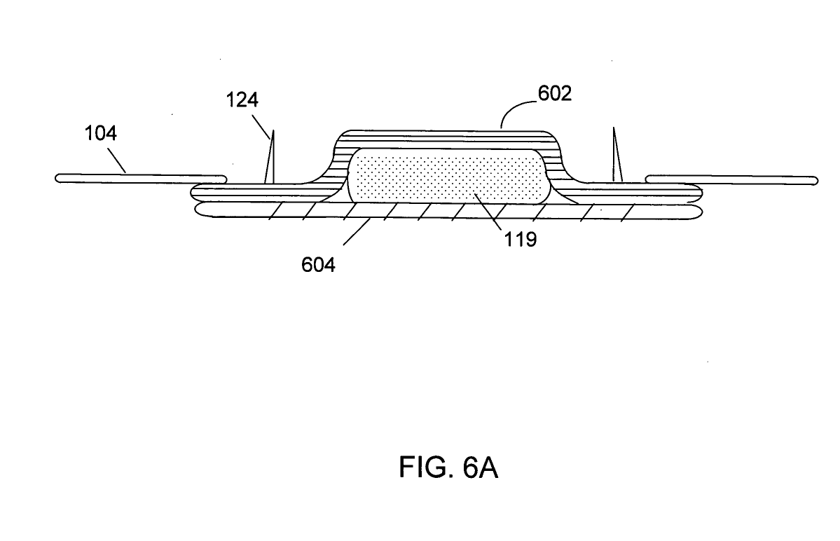

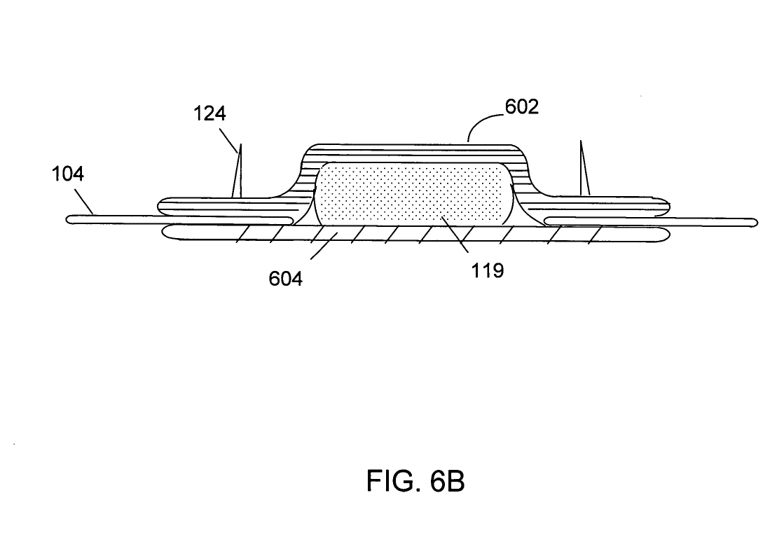

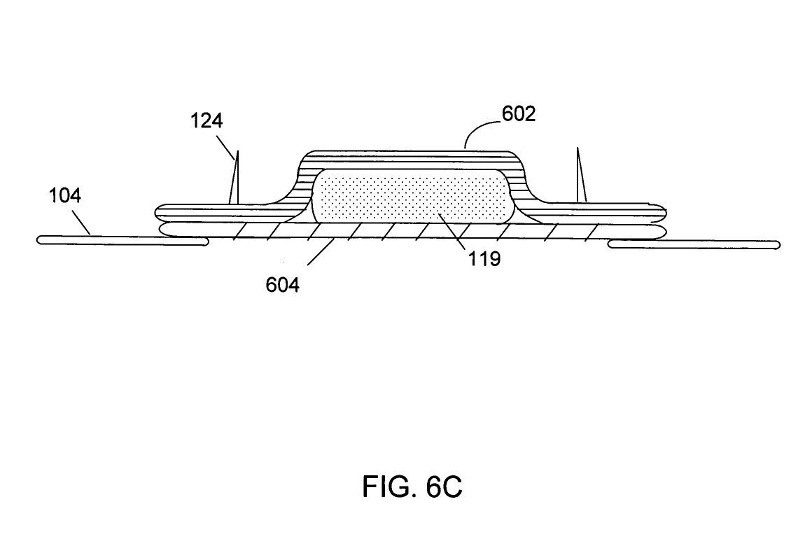

FIGS. 6A, 6B and 6C are detailed cross-sectional views of preferred embodiments of diaper 100 that show the spatial relationship between the various parts of diaper 100. Diaper 100 comprises a top sheet 602, a back sheet 604, and an absorbent core 119 disposed or sandwiched between top sheet 602 and the back sheet 604. In one embodiment, depicted in FIG. 6A, side panel 104 is attached to top sheet 602. In another embodiment, shown in FIG. 6B, side panel 104 is attached to back sheet 604. FIG. 6C shows an embodiment where side panel 104 is sandwiched between top sheet 602 and back sheet 604. It will be apparent that side panel 104 may be attached to diaper 100 using attachment means well known to those skilled in the art such as adhesive, thermal or ultrasonic bonding. Other configurations will be obvious to one skilled in the art based on the teachings provided herein, and the present invention is not limited to these embodiments.

Referring now to FIGS. 1 to 6, top sheet 602 is formed from material such as fibrous non-woven fabric or porous plastic film. Preferred non-woven materials include spun-bonded polypropylene, spun-bonded polyethylene, and thermally bonded webs of staple fibers. Back sheet 604 is made from material such as plastic film or a laminate sheet of plastic film. The plastic film is preferably air-impermeable. Such material should be hydrophobic, soft in texture, and strong in tensile strength. An example includes hydro-entangled non-woven webs, which may contain some cotton and/or rayon fibers blended in with thermoplastic fibers. Cellulose fibers can also be blended in at small percentages to reduce cost. Other materials for forming backsheet 604 may include polypropylene films, co-extruded films (polyethylene and ethylene vinyl acetate), co-polymer films (polyethylene/polypropylene), and polylaminates (polypropylene nonwoven and polyethylene film). Absorbent core 119 can be made up of any suitable absorbent material, as well as combinations of different types of absorbent materials. For example, the absorbent may be formed of a mixture of pulp fluff and superabsorbent particles wrapped in a liquid permeable tissue wrap. The superabsorbent particles may be substantially homogeneously mixed with the hydrophilic fibers or may be non-uniformly mixed. The pulp fluff may be exchanged with synthetic, polymeric, meltblown fibers or with a combination of meltblown fibers and natural fibers. Examples of superabsorbent materials include polyacrylamides, polyvinyl alcohol, polyacrylates, various grafted starches, and the like. A desired super absorbent material is a cross-linked polysodium acrylate, which can be purchased from BASF Corporation of Portsmouth, Va., under the trademark ASAP 2260. Absorbent core 119 may be bonded to top sheet 602 and back sheet 604 with adhesive or welding. Leg elastics 120 and waist elastics 122 comprise material such as thread- or tape-like rubber, tape-like plastic foam or plastic film.

Materials suitable for the side panel 104 of diaper 100 are generally known to those skilled in the art. For example, suitable materials for the side panels 104 include those materials described above as being suitable for the back sheet 604 or top sheet 602 of the diaper 100 such as woven and nonwoven materials or laminates of such materials. Suitable materials include, without limitation, block copolymers of polystyrene, polyisoprene and polybutadiene; copolymers of ethylene, natural rubbers and urethanes; and combinations of the foregoing. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof.

Desirably, the side panels 104 are extensible or elastic to provide improved fit about the wearer. For example, the side panels 104 may comprise a stretch-thermal laminate (STL), neck-bonded laminate (NBL), or stretch-bonded laminate (SBL) material. Methods of making such materials are well known to those skilled in the art and described in U.S. Pat. No. 4,663,220 issued to Wisneski et al., U.S. Pat. No. 5,226,992 issued to Mormon, and European Patent Application No. EP0217032 of Taylor et al., the respective disclosures of which are hereby incorporated by reference in their entireties.

When made with extensible or elastic materials, the side panel 104 is desirably capable of elongating in the lateral direction from about 10 percent to about 400 percent, more desirably at least about 100 percent, even more desirably from about 100 to about 300 percent, and still yet more desirably from about 150 to about 250 percent for improved fit and performance. The stretchability of the side panel when elastic materials are used allows the side panel 104 to stretch over and around the hips of the wearer as the prefastened diaper is pulled on while still maintaining proper fit at the waist after the diaper is correctly positioned on the wearer.

Preferably, side panel 104 is a neck-bonded laminate material for improved manufacturing due to its ability to stretch in the cross machine direction. For example, in a particular embodiment, side panel 104 includes a neck-bonded laminate material which includes a urethane film having a basis weight of about 15 grams per square meter and commercially available from Shawmut Mills as SHAWMUT TX-1560. This material is sandwiched between two layers of necked, stretched spunbond. Each spunbond layer has a basis weight of about 16 grams per square meter and is composed of 3.0 denier polypropylene fibers. The composite is laminated together with an adhesive spray at an add-on rate of about 0.3 grams per square meter. A suitable adhesive is available from Findley Adhesive as FINDLEY 2525A. Such a neck-bonded laminate material is generally capable of elongating in the cross machine direction about 185 percent.

The aforementioned embodiments and examples are meant to be just for explanatory purposes only, and are in no way meant to limit the full scope of the invention. Various other configurations are possible and within the spirit of the invention. The invention is intended to cover all such embodiments within the boundaries defined by the appended claims.

What is claimed is:

1. An absorbent article comprising:
an absorbent chassis comprising a top sheet, a back sheet and an absorbent core disposed between the top sheet and the back sheet, the absorbent chassis defining a front waist region, a back waist region and a crotch region that extends between and interconnects the front and back waist regions;
a non-contoured side panel permanently attached to and extending away from lateral edges of the back waist region of the absorbent chassis; and
at least one fastening component attached to the front waist region of the absorbent chassis;
wherein the side panel extending from the back waist region of the absorbent chassis is engaged to the fastening component on the front waist region to secure the absorbent article to a wearer.

2. The absorbent article as claimed in claim 1 further comprising a pair of leg elastics adapted to fit about the legs of a wearer.

3. The absorbent article as claimed in claim 1 further comprising a pair of waist elastics adapted to fit about the waist of a wearer.

4. The absorbent article as claimed in claim 1 further comprising containment flaps, the containment flaps for reducing the leakage of body exudates from the absorbent article.

5. The absorbent article as claimed in claim 1 wherein the side panel is made of an elasticized material.

6. The absorbent article as claimed in claim 1 wherein the side panel is permanently attached to the absorbent chassis using ultrasonic bonding.

7. The absorbent article as claimed in claim 5 wherein the elasticized material is selected from the group consisting of block copolymers of polystyrene, polyisoprene and polybutadiene, copolymers of ethylene, natural rubber and urethanes, stretchable copolymers of ethylene-propylene, stretch-thermal laminate (STL), neck-bonded laminate (NBL), or stretch-bonded laminate (SBL) material.

8. The absorbent article as claimed in claim 1 wherein the side panel is engaged to the at least one fastening component through a bonding method selected from the group consisting of adhesive, mechanical, thermal, cohesive and ultrasonic bonding.

9. The absorbent article as claimed in claim 1 wherein the at least one fastening component is formed from a loop material and the side panel comprises a hook material which can be releasably engaged to the loop material of the fastening component.

10. The absorbent article as claimed in claim 1 wherein the at least one fastening component is formed from a hook material and the side panel comprises a loop material releasably engaged to the hook material of the fastening component.

11. An absorbent article comprising:
an absorbent chassis comprising a top sheet, a back sheet and an absorbent core disposed between the top sheet and the back sheet, the absorbent chassis defining a front waist region, a back waist region and a crotch region that extends between and interconnects the front and back waist regions;
a non-contoured side panel permanently attached to and extending away from lateral edges of the back waist region of the absorbent chassis; and
at least one fastening component attached to the back waist region of the absorbent chassis;
wherein the side panel extending from the front waist region of the absorbent chassis is engaged to the fastening component on the back waist region to secure the absorbent article on a wearer.

12. An absorbent article comprising:
an absorbent chassis comprising a top sheet, a back sheet and an absorbent core disposed between the top sheet and the back sheet, the absorbent chassis defining a front waist region, a back waist region and a crotch region that extends between and interconnects the front and back waist regions;
a non-contoured side panel permanently attached to and extending away from lateral edges of the back waist region of the absorbent chassis;
at least one fastening component attached to the front waist region of the absorbent chassis;
a pair of leg elastics adapted to fit about the legs of a wearer;
a pair of waist elastics adapted to fit about the waist of a wearer;
wherein the side panel extending from the back waist region of the absorbent chassis is engaged to the fastening component on the front waist region to secure the absorbent article on a wearer.
